(19) United States
(12) Patent Application Publication
(10) Pub. No.: US 2011/0218499 A1
(43) Pub. Date: Sep. 8, 2011

(54) DEVICE FOR INJECTING FLUID FOR MEDICAL USE

(76) Inventor: Jean-Paul Cahen, Montbonnot (FR)

(21) Appl. No.: 13/002,872

(22) PCT Filed: Jul. 7, 2009
(86) PCT No.: PCT/FR2009/051346

§ 371 Date: May 23, 2011

(30) Foreign Application Priority Data
Jul. 7, 2008 (FR) 0803856

(51) Int. Cl. A61M 5/31

(52) U.S. Cl. 604/216

(57) ABSTRACT

The invention relates to a device for injecting a fluid for medical use and is characterized in that the device consists of a base wherein a container (2) for receiving the injection fluid is provided, said container being formed by a bellows-shaped body (6) made of deformable material and including a top surface (4) comprising a tip (7) through which fluid passes, while the base (1) consists of a bottom wall, enabling the axially translatable piston rod to be guided, and a top wall, characterized in that:—the tip (7) is inserted into a suitable arrangement of the lop wall of the base,—the top surface (4) and the bottom wall are conical in shape, while the chamber (6) of the container (2) consists of circular rings, and the piston has a shape complementing the shape of the cavity (8) of the bottom wall (5) such that, when the bellows is completely folded up into itself after injection, the chamber is totally empty, with no residual liquid.

The invention relates to a device for injecting fluid for medical use, intended for the human or animal body.

Current medicine uses various types of injections to dispense a substance into the body of patients for various purposes, such as transfusions or artificial feeding methods, for example, in particular through the blood or the digestive tract (parenteral or enteral nutrition). Methods for analyzing and monitoring the state of patients also use injections of contrast agents. This is the case, for example, of studies such as angiography, for which an iodized substance is injected in the vessels, veins and arteries to opacify them, as well as the irrigated tissues and organs, in order to detect possible abnormalities using radiography. Injections of iodized contrast agents are also used to perform a conventional or digital scan or angiography, the intravenous or arterial injection being carried out in the form of a bolus injection, typically at a rate of 0.5 to 35 mL/s.

Nuclear magnetic resonance imaging (MRI) tests also require injecting a contrast agent, such as Gadolinium, for example.

Various injection devices are known, which are generally comprised of an injector containing the substance to be injected and actuated by an electromechanical or a hydraulic mechanism. The injectors used, for example, have a flexible pouch in which the substance is packaged directly in flexible envelopes. These envelopes are then arranged in a sealed enclosure of the injection device under pressure.

Syringe-type injectors are also used, in which the substance is packaged in the barrel of the syringe, which includes a manually or mechanically actuated movable piston, due to an injection device whose mechanism actuates the piston in axial translation.

Injectors with a flexible bag have the advantage of being capable of being pre-filled with the substance to be injected, which makes them easier to handle and improves asepsis. However, since ejection of the substance requires a pressurizing chamber, this increases the size and weight of the injection devices and lengthens their handling.

Syringe injectors are easier to use but have the disadvantage of being capable of being filled only at the time of use. As a result, handling these injectors, prior to being fitted in the injection device, is more time-consuming and especially presents a greater risk of contamination.

The object of the invention is therefore to propose an injector which combines the advantages of the two aforementioned types, without having their disadvantages.

To this end, the object of the present invention is a device for injecting a fluid for medical use, comprising a sleeve in which a container for receiving the fluid to be injected is arranged, such container being comprised of an axially deformable body, of a top surface provided with a tip engaged in a suitable arrangement at the top of the sleeve, for passage of the fluid, and of a bottom surface taking support on a piston movable in axial translation in the sleeve.

The container of the invention is therefore a deformable syringe without piston, since its deformation from a deployed position to a collapsed position occurs due to the deformation of its wall, which is comprised of a succession of deformable circular rings capable of collapsing on themselves.

According to other advantageous characteristics of the invention:
The inner wall of the top of the sleeve and the outer wall of the top surface of the container have complementary shapes.
Similarly, the outer wall of the bottom of the container and the piston have complementary shapes.
Preferably, the top surface and the bottom of the container have a conical shape that is adjusted to enable said bottom to be nested in the top surface at the end of the axial translational movement of the piston.
The volume generated by the conical surface of the bottom of the container forms a hollow outer housing into which the complementary shape of the piston gets nested.
The bottom of the container comprises a stud in its center, which is axially aligned with the tip borne by the top surface and whose diameter, except for the functional clearance, is substantially equal to the inner diameter of the tip. As a result of this complementary geometry, there is zero residual volume at the end of the injection.
The sleeve comprises a window through which the container passes, and its top is provided with a notch that opens out in the window, on the one hand, and in a central opening of the top, which is suitably sized to receive and retain the tip of the container, on the other hand.
The wall of the container body is in the form of a bellows whose profile enables it to collapse during compression.
In an alternative embodiment, the wall of the container body is formed of a bellows having circular rings.
Advantageously, the lateral rigidity of the structure of the container is adjusted to prevent any parasitic elasticity.
The geometry of the bellows is such that deformations other than those generated by the axial translation are quasi nonexistent.
To enable the container to be filled, the piston is provided with a device that enables it to be affixed to the bottom of said container, when the latter is empty, and it is actuated by an axial translational tractive movement of the latter.
In an embodiment, the sleeve can be doubled so as to receive a plurality of containers, each of the containers being controlled independently.

Other characteristics and advantages of the invention will become clearly apparent upon reading the description that follows, given by way an example, with reference to the annexed drawings, in which:

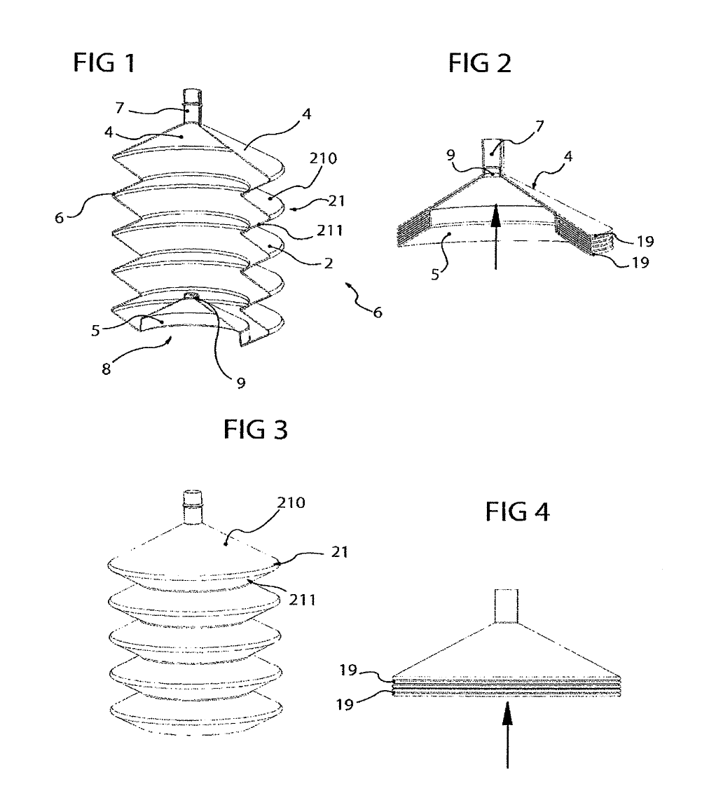

FIG. 1 is a perspective cross-sectional view of the container according to the invention, in the deployed position;

FIG. 2 is perspective cross-sectional view of the same container in the collapsed position;

FIGS. 3 and 4 are views similar to the preceding Figures but according to external view without tear-out;

FIG. 5 is a perspective view of the injection device being used;

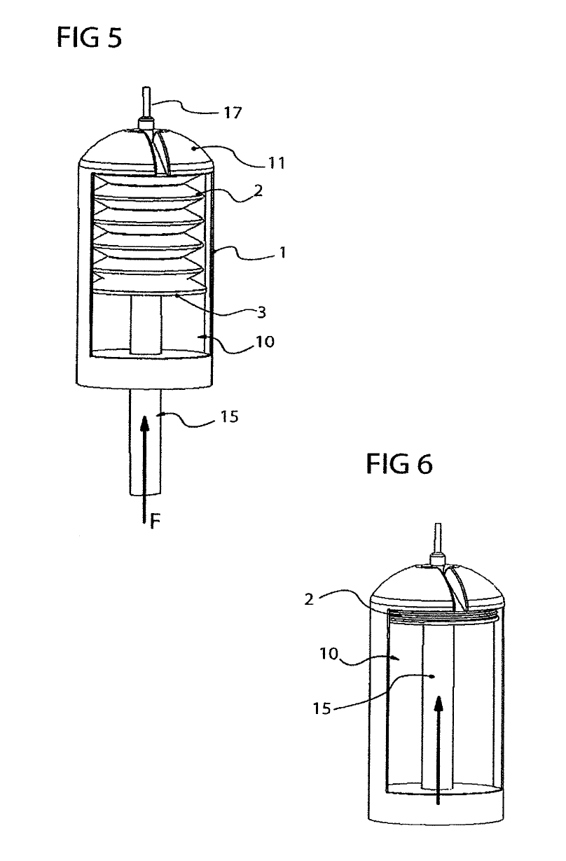

FIG. 6 is a perspective view of the injection device, at the end of the injection, the container being in the completely collapsed position;

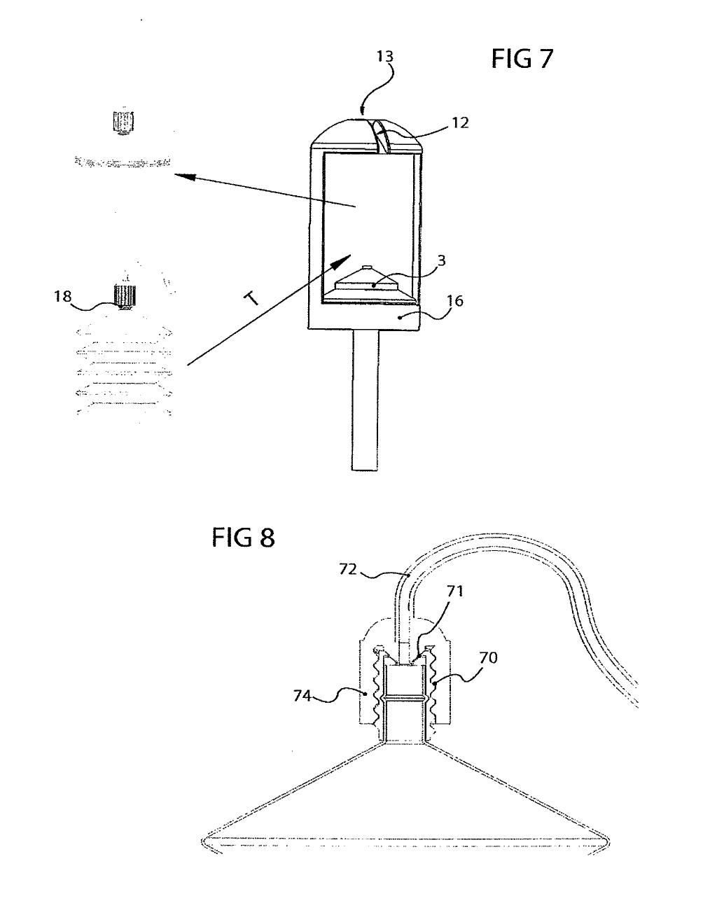

FIG. 7 is a view of the injection device showing the handling of the container; and

FIG. 8 is a detailed view of an improvement.

The injection device according to the invention is comprised (FIG. 5) of a support (1) which, according to the embodiment illustrated, is a generally cylindrical sleeve carrying a container (2) adapted to receive the fluid to be injected. The support (1) includes a piston (3) movable in axial translation.

The container (2) is deformable and is made of a deformable material such as plastic, for example polypropylene, enabling it to be pre-filled with the fluid to be injected.

This container (2) forms a volume whose wall is axially deformable. To this end, the wall is comprised of a top portion (4) and a bottom portion (5), both having the shape of a cone. The peripheral wall forming the body of the enclosure (6) has the shape of a deformable bellows having circular rings (21) such that, when the bellows is completely collapsed on itself, the container is completely empty, with no residual liquid. Thus, the wall of the bellows-shaped container is comprised of a succession of circular rings whose periphery has a triangular cross-section, each of the rings thus including a top surface (210) that is conical upward and a bottom surface (211) that is conical downward. It is noted that in the collapsed position, each top surface (210) is in contact with the bottom surface (211) of the adjacent ring.

It is noted that the top surface (4) of the container, in its center, carries a cylindrical tip (7) through which the fluid contained in said container passes.

The volume generated by the conical surface of the bottom (5) is outside of the volume of the container and thus forms a hollow housing (8). It is noted that the bottom (5), in its center, comprises a centering stud (9) axially aligned with the tip (7), and whose diameter, except for the freedom of axial clearance, is substantially equal to the inner diameter of the tip so that, in the collapsed position, said stud is engaged in the tip, as is illustrated in FIG. 2.

The support (1) comprises an adequately sized lateral opening (10) forming a window to enable the passage and positioning of the container (2), as illustrated in FIG. 5. According to the embodiment shown, the support is a sleeve closed in its upper portion by a top (11) having the shape of a spherical cap on the outside, and the inner walls of which form a conical surface whose shape is complementary to the outer wall of the top portion (4) of the container.

The top (11) comprises a radial notch (12) that opens out in the window (10), on the one hand, and in a central opening (13) of the top, on the other hand.

A piston (3) affixed to one end of a pin (15) is housed in the sleeve (1), such pin extending through the base (16) of the sleeve in order to be connected via its second end to an actuation mechanism capable of generating an axial translational movement F transmitted by the pin (15) to the piston (3).

According to a preferred embodiment of the invention, the outer shape of the piston is complementary to that of the surface opposite the housing (8) formed in the bottom (5) of the container.

It is noted that the support (1), which has the shape of a sleeve according to the illustrations proposed by way of an example, could have any other shape. Thus, it may not have a peripheral wall, and thus be comprised of a stirrup including an upper wall forming the top (11) connected to a lower wall forming the base (16) by one or more connecting arms, or even any other connection.

The method for positioning the container in the support (1) is easily understood from the preceding description. The container (2) containing the medical fluid is inserted therein, through the window (10) along a transverse translational movement. In this step, the piston (3) is in the low position in which it rests on the base (16) of the support.

The tip (7) of the container, provided with a nozzle (17) attached to the tip by means of a connection (18), is inserted via the notch (12) into the opening (13) of the cap (11). Blocking means (not shown), adapted to prevent the container from pivoting about its axis, can be provided.

The piston (3) and the hollow housing (8) of the bottom (5) of the container (2), having complementary shapes, nest completely one into the other, thus blocking the transverse displacement of the latter.

When the actuating mechanism exerts a translational movement along (F) on the pin (15) of the piston, along the longitudinal axis, said piston moves in the same direction by exerting a compressive force on the container, which generates the contraction, and therefore the collapsing and contraction of the deformable wall (6) of the container and, at the same time, the backflow of the fluid contained therein.

It is noted that the complementarity of the shape of the piston and of the bottom of the container enables a uniform collapsing of the body of the container, the collapsing rings (19) nesting in one another, as is illustrated in FIGS. 2 and 4.

When the ejection of the fluid is complete, and the container is completely contracted, the bottom (5) and top surface (4) of the container are in contact with one another, and its stud (9) is engaged in the tip (7) in order not to leave any quantity of residual fluid. In fact, the amount of residual fluid is close to zero, due to the adapted shape of each of the co-operating elements, which makes it possible to obtain completely jointed collapsing rings 19 at the end of the translational movement.

The empty container is then removed from the sleeve along a translational movement opposite that which enabled its insertion therein.

It is noted that the conical shape of the container prevents air bubbles, which can form when it is being filled, from sticking to its walls. These air bubbles are thus located at the top and are evacuated first during the bubble removal operation.

It is noted that the lateral rigidity of the structure of the container is adjusted to avoid any parasitic elasticity within the limit of normal pressures for this type of equipment (21 bars, 300 psi) and that the container is tightly retained in its sleeve, with the exception of the axial displacement clearance.

Indeed, the thrust exerted on the container by the piston must generate a continuous forward movement. To this end, the deformation of the container must be exerted only in the axial direction, i.e., in the direction of the force F generated by the piston in order to control the instantaneous volume and, consequently, the ejection rate and pressure of the fluid contained in said container.

The mechanism for actuating the piston is selected suitably to generate a continuous and adjustable translational movement, such as a hydraulic cylinder or an electric motor, with brushless electronic control, for example.

The container, once emptied, can be filled again. To this end, the sleeve that has just been described, or a similar sleeve, is used. The container is in the position shown in FIG. 6, in which it is pressed flat against the inner wall of the top (11) of the sleeve, its tip (7) being retained in the opening (13) of the top.

The piston is provided with a device enabling it to be affixed to the bottom of the container, and its actuation mechanism exerts an axial translational movement on it, in the opposite direction in relation to the preceding direction (F). The bottom (5) of the container is provided with a means for fastening to the piston (3), thus enabling the filling by suction in the same manner as a conventional piston syringe. The bottom (5) of the container is driven towards the base of the sleeve by the piston, so that the container gradually reassumes its initial shape while sucking the liquid.

It is further noted that the sleeve can be adapted so as to receive a plurality of containers, each of the containers being controlled independently (a plurality of sleeves).

The injection device that has just been described makes it possible, with the same injection system, either to use containers that have been prefilled with the fluid to be injected, or to fill up the containers at the time of use.

It is therefore particularly flexible and versatile. Moreover, its arrangement and its injection system are simple, and it does not require a power generator that is penalizing in terms of cost and space requirement.

FIG. 8 is a detailed view of an improvement according to which the tip (7) includes a stopper (70) equipped with a cover (71), whereas, during connection to the injection tube (72), a projection (73) of the connector (74) perforates the cover (71).

It is understood that when the container (2) is completely compressed at the end of the injection, the rings forming the bellows are jointed via tight nesting, so that there is no residual medical liquid left.

It is also understood that the bottom wall (16) is used to guide the piston rod, whereas the top wall (11) is used to support the enclosure (2).

1. Device for injecting a fluid for medical use, characterized in that it is comprised of a support (1) in which a container (2) for receiving the fluid to be injected is arranged, said container being formed of a body (6) made a of deformable material, having the shape of a bellows in order to be capable of being axially compressed and including a top surface (4) comprising a tip (7) through which the fluid passes, whereas the support (1) is comprised of a bottom wall (16) enabling the guiding of the rod of a piston (3) movable in axial translation, and of a top wall (11), characterized in that
the tip (7) is engaged in a suitable arrangement (12), (13) of the top wall (11) of the support,
the top surface (4) and the bottom wall (16) have the shape of a cone, whereas the enclosure (6) of the container (2) is comprised of circular rings, and the shape of the piston (3) is complementary to the shape of the housing (8) of the bottom wall (5), such that when the bellows is completely collapsed on itself after injection, the enclosure is empty, with no residual liquid.

2. Injection device according to claim 1, characterized in that the wall of the container is comprised of a succession of circular rings whose periphery is triangular in cross-section, each of the rings thus including a top surface (210) that is conical upward and a bottom surface (211) that is conical downward, so that, in the collapsed position, each top surface (210) is in contact with the bottom surface (211) of the adjacent ring.

3. Injection device according to claim 2, characterized in that the enclosure (2) is made out of a plastic material.

4. Injection device according to claim 3, characterized in that the bottom (5) comprises, in its center, a stud (9) that is axially aligned with the tip (7) carried by the top surface (4), and whose diameter, except for the axial clearance, is substantially equal to the inner diameter of the tip.

5. Injection device according to claim 1, characterized in that the support (1) comprises a window (10) through which the container passes, and in that its top (11) is provided with a notch (12) that opens out in the window (10), on the one hand, and in a central opening (13) of the top, which is suitably sized to receive and retain the tip (7) of the container, on the other hand.

6. Injection device according to claim 1, characterized in that the piston (3) is provided with a device enabling it to be affixed to the bottom (5) of the container, when the latter is empty, and in that it is actuated by of an axial translational tractive movement of the latter.

7. Injection device according to claim 1, characterized in that the support is adapted so as to receive a plurality of containers, each container being controlled independently.

8. Injection device according to claim 1, characterized in that the containers ready for use, whether empty or full, are equipped with a pierceable tip guaranteeing the sterility of the unit.
